(19) United States
(12) Patent Application Publication
(10) Pub. No.: US 2016/0199149 A1
(43) Pub. Date: Jul. 14, 2016

(54) Alignment Mark

(71) Applicant: Reference Technology Limited Company, Taichung City (TW)

(72) Inventors: Sheng-Hong Tseng, Taipei City (TW); Ching-Te Chen, New Taipei City (TW); Ya-Yu Tsou, Taichung City (TW); Chun-Hwei Tai, New Taipei City (TW)

(21) Appl. No.: 14/984,161

(22) Filed: Dec. 30, 2015

(30) Foreign Application Priority Data
Jan. 8, 2015 (TW) 104200283

(51) Int. Cl. A61B 90/00

(52) U.S. Cl. CPC A61B 90/39; A61B 2090/3966; A61B 2090/3954; A61B 2090/3937

(57) ABSTRACT

An alignment mark is adapted to be positioned relative to a head of a patient, and includes an alignment unit and a marking unit. The alignment unit includes a pad member and an alignment member. The pad member is adapted to adhere to a scalp of the patient. The alignment member has a threaded section that extends through the pad member and that is adapted to be threadedly locked into a cranium of the patient, and a head section that is opposite to the threaded section and that abuts against the pad member. The marking unit is removably disposed on the alignment unit, and includes a marking member.

### CROSS-REFERENCE TO RELATED APPLICATION

This application claims priority of Taiwanese Application No. 104200283, filed on Jan. 8, 2015.

### FIELD

The disclosure relates to an alignment mark, more particularly to a miniature three-dimensional alignment mark which is adapted to be applied in medical operations.

### BACKGROUND

Nowadays, most doctors perform an operation with the assistance of medical imaging and computer technology. When generating medical images, a plurality of alignment marks are positioned on a head of a patient and scanned by a computed tomography (CT) machine or a magnetic resonance imaging (MRI) machine to confirm a position of a lesion. This allows for appropriate surgical planning, such as whether the operation should be performed via a conventional approach or through Stereotactic Functional surgery with a surgical navigation system.

When performing the Stereotactic Functional surgery, the medical images are inputted into the surgical navigation system to rebuild a three-dimensional image of the head and confirm the position of the lesion. The alignment marks on the patient's head are regarded as reference coordinate points that assist the surgical navigation system in determining both surgical instrument and lesion positions during operation. Consequently, the doctor is able to control the medical machines to avoid important regions in the head while treating the lesion.

Therefore, the alignment mark plays a crucial role in generating medical images, including confirming the position of the lesion, planning the surgery, and performing the CAS.

As shown in FIG. 1, a conventional alignment mark 1 length is between 7 and 13 millimeters. The conventional alignment mark 1 has a threaded section 11 that can be threadedly locked into a human cranium, a marking section 12 opposite to the threaded section 11, and a body section 13 connected between the threaded section 11 and the marking section 12. Due to hardness of the human cranium, a user needs to use a tool that can offer a stable torque for installing the alignment mark 1.

An adult cranium has a thickness between 8 and 9 millimeters, while the cranium of a child has a thickness between 2 and 3 millimeters. Hence, the size of the conventional alignment mark 1 may be too big for the child, resulting in cranium fracture when the threaded section 11 is threadedly drilled into the child's cranium. However, it is relatively difficult to manufacture the conventional alignment mark 1 that is reduced in size. Even if the size of the conventional alignment mark 1 is reduced, detection by the surgical navigation system (usually the size of the alignment mark 1 has to be larger than 2 millimeters) would be rather difficult. Furthermore, the cranium of the child is relatively soft and cannot offer a suitable resistance to prevent overdrilling of the conventional alignment mark 1.

In addition, an adult patient having a thinner cranium is also not suitable for treatment with the conventional alignment mark 1. As a result, an alignment mark that can be widely applied is needed in the market.

### SUMMARY

Therefore, the object of the disclosure is to provide an alignment mark which is adapted to be positioned relative to a thin cranium of an adult or a cranium of a child.

Accordingly, an alignment mark of the present disclosure is adapted to be positioned on a head of a patient, and includes an alignment unit and a marking unit. The alignment unit includes a pad member and an alignment member. The pad member is adapted to adhere to a scalp of the patient. The alignment member has a threaded section that extends through the pad member and that is adapted to be threadedly locked into a cranium of the patient, and a head section that is opposite to the threaded section and that abuts against the pad member. The marking unit is removably disposed on the alignment unit and includes a marking member.

### BRIEF DESCRIPTION OF THE DRAWINGS

Other features and advantages of the disclosure will become apparent in the following detailed description of the embodiment with reference to the accompanying drawings, of which:

FIG. 1 is a side view of a conventional alignment mark;

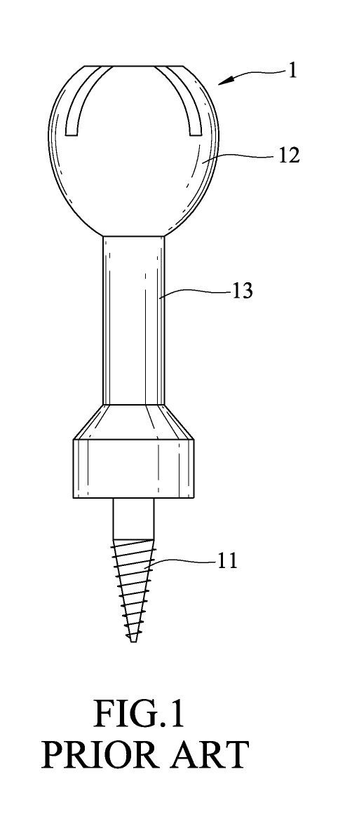

FIG. 2 is an exploded perspective view of an embodiment of an alignment mark according to the disclosure;

FIG. 3 is a fragmentary exploded perspective view of the embodiment illustrating an alignment member ready to be threadedly locked into a human cranium with a screwdriver; and

FIG. 4 is a partly exploded perspective view of the embodiment illustrating a marking unit ready to be coupled to an alignment unit.

### DETAILED DESCRIPTION

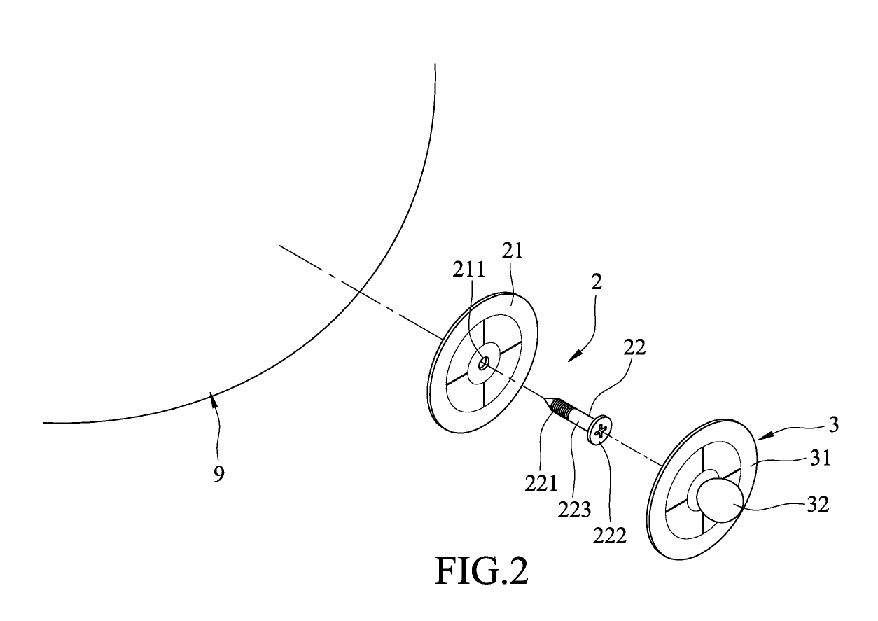

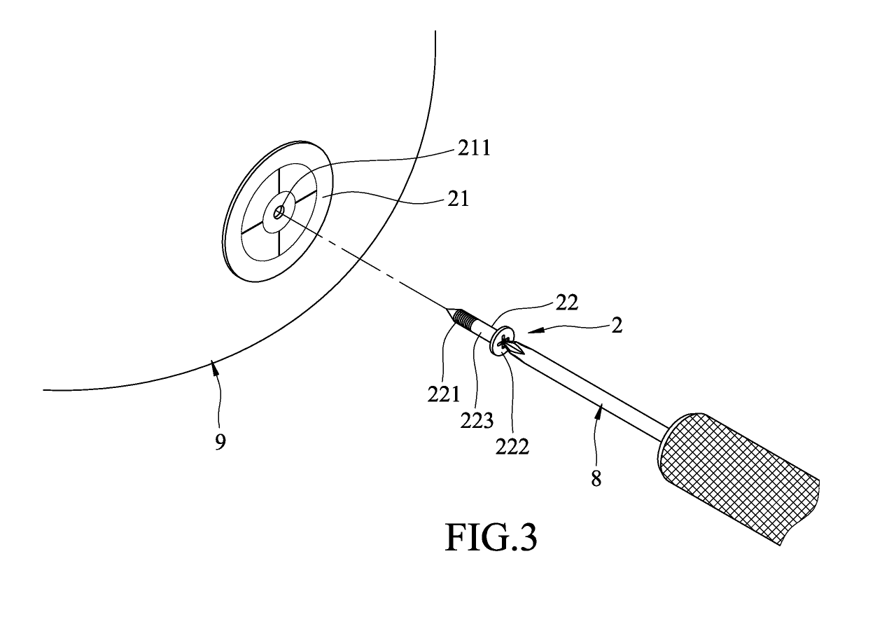

As shown in FIGS. 2 and 3, the embodiment of an alignment mark according to the present disclosure is adapted to be positioned on a head 9 of a patient. The alignment mark includes an alignment unit 2 and a marking unit 3.

The alignment unit 2 includes a pad member 21 and an alignment member 22. The pad member 21 is adapted to adhere to a scalp of the patient, and has a through hole 211 formed at a center thereof. The alignment member 22 has a threaded section 221 that extends through the through hole 211 of the pad member 21 and that is adapted to be threadedly locked into a cranium of the patient, a head section 222 that is opposite to the threaded section 221, and a body section 223 connected between the threaded section 221 and the head section 222.

When positioning the alignment unit 2 on the head of the patient, a user first places the pad member 21 on a desired position on the scalp of the patient, and then locks the alignment member 22 into the cranium of the patient using a screwdriver 8. Specifically, the threaded section 221 is threadedly extended through the through hole 211 of the pad member 21 and drilled through the scalp and into the cranium of the patient until the head section 222 abuts against the pad member 21. At this time, the pad member 21 fixedly adheres to the scalp of the patient and prevents overdrilling of the threaded section 221.

The alignment member 22 is independently manufactured such that a size thereof can be varied depending on requirements of different patients. For example, the size of the alignment member 22 may be reduced for avoiding injuries to a child patient whose cranium is thin.

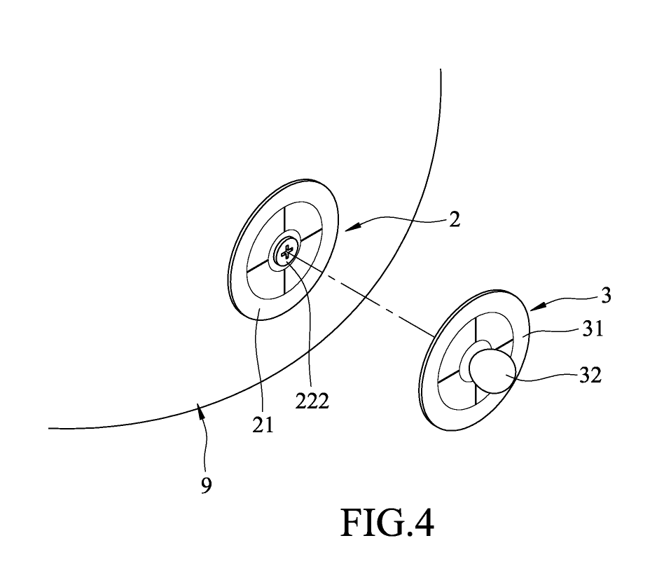

Referring to FIGS. 2 and 4, the marking unit 3 is removably disposed on the alignment unit 2, and includes a connecting member 31 and a marking member 32. The connecting member 31 is removably disposed on the alignment unit 2. The marking member 32 is formed as a ball, disposed on the connecting member 31, and aligned with the alignment member 22. In this embodiment, the connecting member 31 is disposed on the alignment unit 2 and is configured as a pad having the same shape as the pad member 21 of the alignment unit 2. The marking member 32 is disposed on a central axis of the alignment member 22.

The marking member 32 is independently manufactured such that the size thereof is controllable. The size of the marking member 32 can be enlarged for generation of medical images and is not influenced by the size of the alignment member 22.

The alignment member 22 is threadedly locked into the human cranium such that shifting of the scalp of the patient, which would affect disposition precision of the marking unit 3, can be avoided in an operation.

In conclusion, with the configuration of the alignment mark according to the disclosure, the size of the alignment member 22 may vary depending on a thickness of the cranium of the patient or a location of a lesion, and an injury to the cranium of the patient can be avoided.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. An alignment mark adapted to be positioned on a head of a patient, comprising:
an alignment unit including
a pad member adapted to adhere to a scalp of the patient, and
an alignment member having a threaded section that extends through said pad member and that is adapted to be threadedly locked into a cranium of the patient, and a head section that is opposite to said threaded section and that abuts against said pad member; and
a marking unit removably disposed on said alignment unit, and including a marking member.

2. The alignment member as claimed in claim 1, wherein said marking unit further includes a connecting member removably disposed on said alignment unit, said marking member being disposed on said connecting member and aligned with said alignment member.

3. The alignment mark as claimed in claim 2, wherein said pad member has a through hole formed at a center thereof for extension of said threaded section of said alignment member therethrough.

4. The alignment mark as claimed in claim 2, wherein said marking member is formed as a ball, and is disposed on a central axis of said alignment member.

5. The alignment mark as claimed in claim 2, wherein said connecting member is configured as a pad having the same shape as said pad member of said alignment unit.
